(19) United States
(12) Patent Application Publication
(10) Pub. No.: US 2016/0193002 A1
(43) Pub. Date: Jul. 7, 2016

(54) SURGICAL APRON SHEET

(71) Applicants: Osamu Motoyama, Tokyo (JP); Merio Co., Ltd., Tokyo (JP)

(72) Inventor: Osamu Motoyama, Tokyo (JP)

(21) Appl. No.: 14/901,285

(22) PCT Filed: Jul. 3, 2013
(86) PCT No.: PCT/JP2013/004139

§ 371 Date: Dec. 28, 2015

(51) Int. Cl. A61B 46/20

(52) U.S. Cl. CPC A61B 46/20

(57) ABSTRACT

A surgical apron sheet to cover the face to prevent eye inflammation and the like caused by, inter alia, scattering oral bacteria, while also facilitating treatment of the oral cavity. The surgical apron sheet has a structure that resists slipping when covering the face during treatment of the oral cavity and is easy to manufacture at low cost. The surgical apron sheet of apron shape includes a face covering portion to cover the face of a patient during treatment and provided with a mouth-like opening. The opening is provided at a lower end with an engaging portion cut as a long hole of substantially semi-elliptic shape to engage a submental region or lower jaw line of the patient being treated.

### TECHNICAL FIELD

The present invention relates to a sheet used chiefly during dental treatment that doubles as a surgical protective cover for covering the face and body of a patient, particularly to a surgical apron sheet of slip-resistant structure for use during oral treatment to cover face and clothing in order to prevent eye inflammation and the like caused by, inter alia, scattering of oral bacterial by covering the face, to protect clothing from scattering surgical water and liquid medicines, and to facilitate easy and anxiety-free oral treatment.

### BACKGROUND OF THE INVENTION

Various sheets for covering a patient to protect the patient under surgery from matter scattered in the course of an operation have been developed and are used at the time of treatment. Among such cases, scattering of liquid medicines and other substances is particularly abundant in dental treatment because the method of treatment involves direct drilling of teeth under treatment with dental equipment, and since the scattered matter sticks to clothing and especially because it causes inflammation on entering the eyes, there is an urgent need to take measures to protect the patient against scattering substances.

Techniques relating to such sheets for protecting a patient during treatment include that disclosed in Japanese Patent Publication (A) No. 2001-46407, for example. Here there is disclosed a structure having a window hole the size of a human mouth shape formed near the center of a towel. A method of performing dental or oral treatment/measures in a state with the dental or oral towel placed lengthwise over the person's face and with the mouth exposed through the window hole and a method of performing dental or oral treatment/measures in a state with the towel placed across the face with the opposite ends folded forward to overlap corner portions are disclosed. It is suggested that this makes it possible to prevent scattered water droplets, liquid medicine and soiling matter from adhering to clothing, face, head and so on.

However, in accordance with the disclosed invention, the covered region of the patient's face varies depending on how the towel is overlaid, so that it has a drawback of not being able to achieve its purpose of reliably protecting the eyes. In addition, the opening position shifts when the patient moves the mouth during treatment, and since this make it necessary to adjust the position of the opening at every instance, a problem arises of it being difficult to concentrate efficiently on the treatment.

Moreover, Japanese Patent Publication (A) No. 2007-181665 discloses a surgical apron (drape) sheet used by placing it on the face of the patient being treated, and discloses technical concepts of a structure whereby, when placed over the face, an edge region of a mouth portion provided on the sheet extends from the vicinity of the nasolabial folds to surround the mentum or chin, a configuration wherein a lower edge of the mouth portion lies in the vicinity of the mentolabial sulcus between the lower lip and the mentum, and a configuration wherein an upper end of the sheet is placed to lie near the hairline of the patient being treated. By this, even if the sheet is placed on the face of the patient being treated, a somewhat widely opened oral cavity can be seen from the exterior, enabling treatment and the like to be performed unhindered, while it is also suggested that the lower edge of the opening can latch into the concave mentolabial sulcus and that vertical slipping of the sheet position can be prevented by the edge hooking on the nose.

However, disadvantages include that when the mouth is opened wide as required in the course of an operation, the lower edge of the opening detaches and enters the open mouth or covers the mouth, making the oral cavity hard to see during the operation.

According to the invention disclosed here, since it is chiefly the lower lip region that holds the sheet in place, the patient being treated unconsciously becomes anxious about keeping the sheet from shifting, so that the patient's mental stress has by no means been small. In addition, it is impossible to prevent the sheet form slipping when the person under treatment makes a big mouth movement, so that in actuality it has been impossible to reliably keep the sheet from shifting during treatment that involves pain.

Development has therefore been desired of a surgical apron sheet that can be fastened to resist shifting no matter how the patient being treated moves the mouth, reliably prevents the patient being treated from scattered matter such as liquid medicines during treatment, and is easy to manufacture and low in cost.

Patent Document 1: Japanese Patent Publication (A) No. 2001-46407

Patent Document 2: Japanese Patent Publication (A) No. 2007-181665

### OBJECT AND SUMMARY OF THE INVENTION

### Problems to be Overcome by the Invention

In order to overcome the aforesaid problems, the present invention has as its object to provide an apron sheet exhibiting surgical drape performance for use chiefly during dental treatment to cover the face and body of a patient undergoing treatment, particularly a surgical apron sheet for covering the face to prevent eye inflammation and the like caused by scattering of oral bacterial etc., which has a slip-resistant structure for facilitating treatment in the oral cavity when the oral cavity is treated with the face covered, and is also easy to manufacture and low in cost.

### Means for Solving the Problem

In order to achieve the aforesaid object, the surgical apron sheet according to the present invention is a surgical apron sheet of apron shape at least one surface of which is waterproofed and/or coated with a waterproof membrane, which surgical apron sheet comprises a face covering portion for covering a patient's face during treatment and is provided with a mouth-like opening for performing oral cavity treatment, wherein the opening is provided at a lower end with an engaging portion cut to substantially semielliptic shape to engage a submental region or lower jaw line of the patient being treated in order to immobilize the face covering portion.

Moreover, the surgical apron sheet comprises a trunk covering portion provided at a lower end of the face covering portion for covering the trunk of the patient being treated.

Further, the opening is configured to engage along the patient's nose bridge by providing a nose bridge engaging portion constituted of a slit and/or cutout incised upward from the opening edge to separate left-right from the peak of the nose contour.

Further, in another configuration, the trunk covering portion is formed with shoulder portions of greater breadth than the face covering portion and fitting straps for fastening the main sheet to a person's body are integrally attached partially detachably to the shoulder portions.

### Effects of the Invention

Being configured in the foregoing manner, the present invention offers the following effects.

1. Since the opening is provided at the lower end with an engaging portion cut to substantially semielliptic shape to engage the submental region or lower jaw line of the patient being treated, the lower end of the opening can be reliably fitted/engaged on the submental region or lower jaw, so that the face covering portion can be properly immobilized with no slipping or detachment of the engagement due to mouth movements of the patient being treated.

2. Owing to the provision of the trunk covering portion at the bottom end of the face covering portion, the trunk of the patient being treated can be covered and the patient's skin and clothing protected against contamination by oral bacteria and the like contained in liquids and other matter scattered during treatment.

3. A slit and/or cutout by which the face covering portion separates left-right from the peak of the nose contour is provided at the upper end of the opening as the nose bridge engaging portion constituted in a shape formed by cutting upward from the upper end of the opening, whereby slipping of the surgical apron sheet can be prevented because the face covering portion engages along the nose bridge of the patient being treated.

4. As shoulder portions of greater breadth than the face covering portion are formed on the trunk covering portion, the shoulder portions can be fastened to the patient being treated. Further, owing to the adoption of the structure having the partially detachable fitting straps integrally provided on the shoulder portions, the main sheet can be easily fitted or engaged/fastened on the trunk of the patient being treated, while it additionally becomes possible to form long fitting straps integrally with the main sheet, so that the surgical apron sheet can be configured to be usable without waste, easily manufactured and low in cost, with no need to stock strips or the like.

### BRIEF EXPLANATION OF THE DRAWINGS

FIG. 1a is a front view of a surgical apron sheet of the present invention.

FIG. 1b is a front view of the surgical apron sheet provided with a trunk covering portion.

FIG. 2a is a front view showing the surgical apron sheet in use.

FIG. 2b is a front view showing the surgical apron sheet before use.

FIG. 3 is a front view of the surgical apron sheet provided with fitting straps.

FIG. 4 is a front view of the surgical apron sheet provided with long fitting straps or a looped strap.

FIG. 5 is a front view showing another embodiment of the surgical apron sheet.

### DETAILED DESCRIPTION OF THE EMBODIMENTS

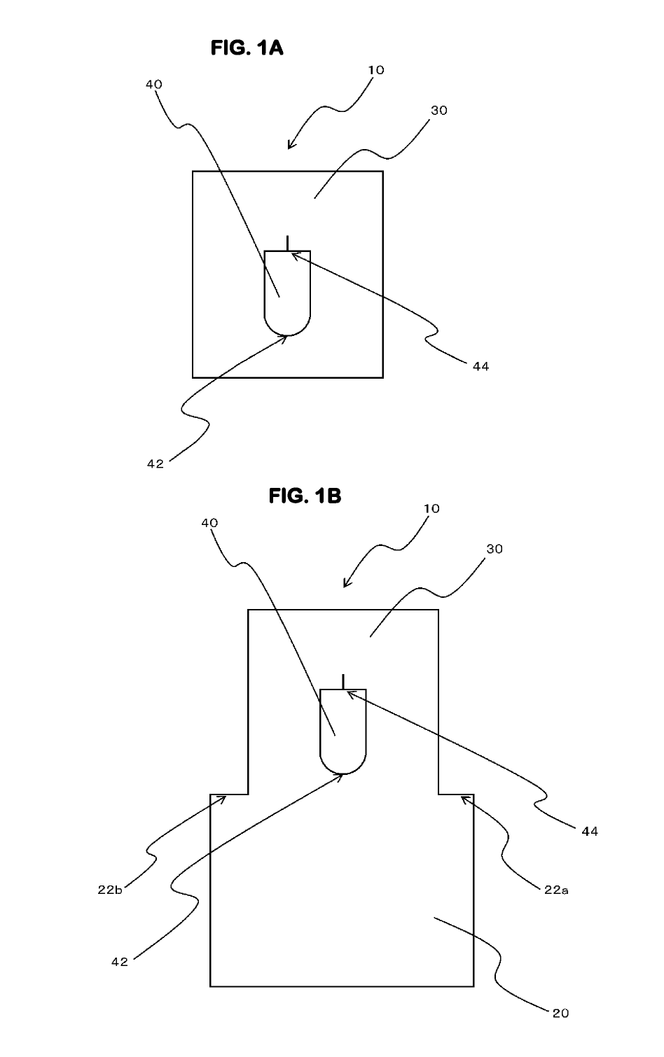

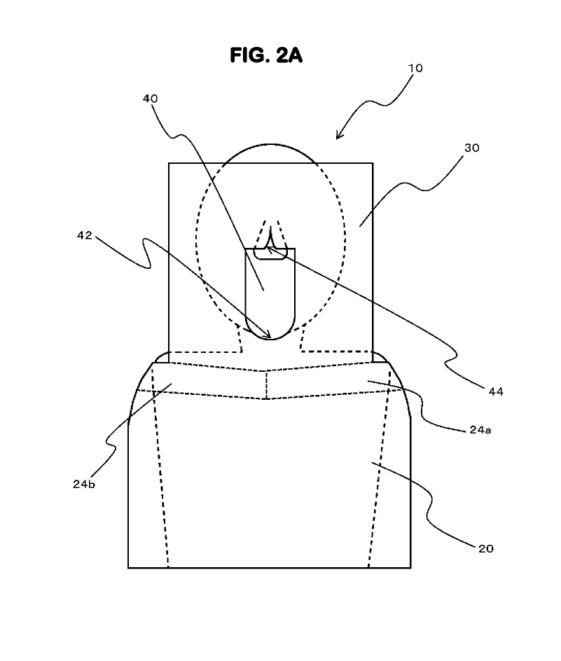

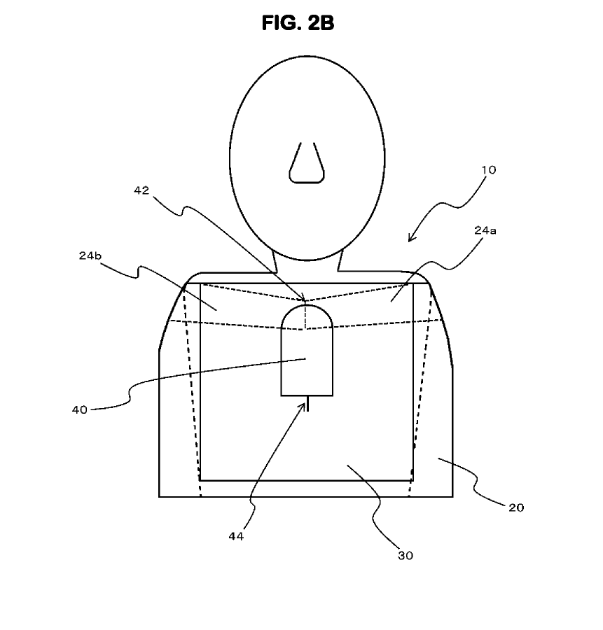

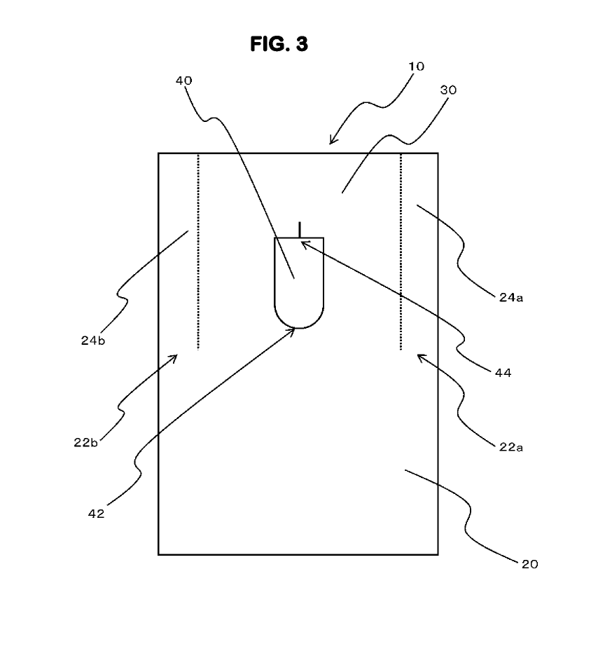

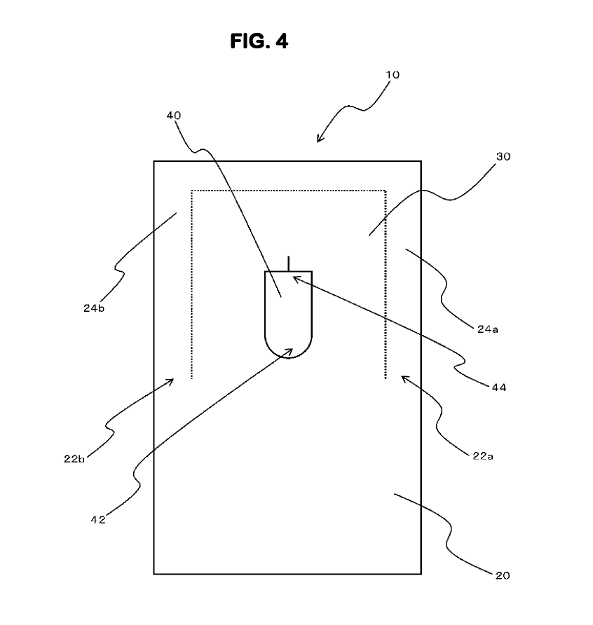

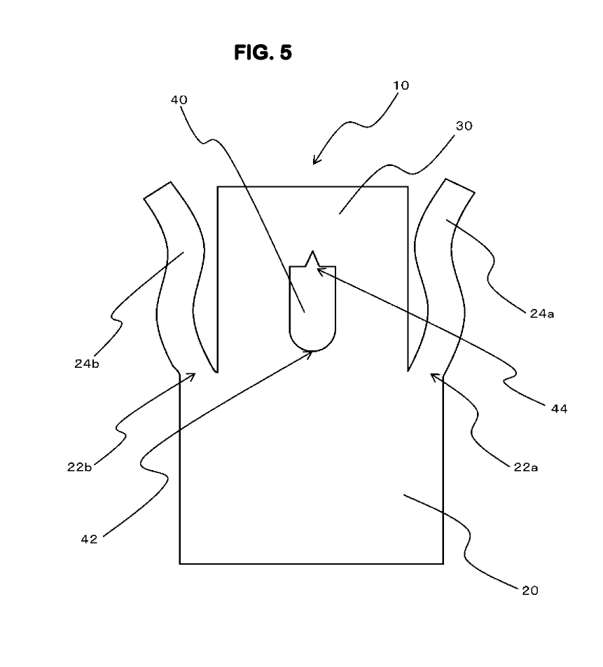

The surgical apron sheet according to the present invention is explained in detail below based on embodiments shown in the drawings. FIG. 1a is a front view of a surgical apron sheet of the present invention, and FIG. 1b is a front view of the surgical apron sheet provided with a trunk covering portion. FIG. 2a is a front view showing the surgical apron sheet in use, and FIG. 2b is a front view showing the surgical apron sheet before use. FIG. 3 is a front view of the surgical apron sheet provided with fitting straps, and FIG. 4 is a front view of the surgical apron sheet provided with long fitting straps or a looped strap. FIG. 5 is a front view showing another embodiment of the surgical apron sheet.

As shown in FIG. 1a, the surgical apron sheet of the present invention comprises a main sheet 10, a face covering portion 30, and an opening 40. Further, as shown in FIG. 1b, it can be configured to comprise the main sheet 10, a trunk covering portion 20, the face covering portion 30, and the opening 40.

As shown in FIG. 1a and FIG. 1b, a configuration is adopted that comprises an apron-shaped sheet, at least one surface of which is waterproofed and/or coated with a waterproof membrane. This configuration makes it possible to protect against soiling of the patient's clothing by oral bacteria etc. that scatter onto and might pass through the main sheet 10 during treatment and to prevent various germs contained in the permeating saliva from contacting and entering the eyes and so on of the patient being treated.

Another aspect is that the main sheet 10 can be made of a liquid absorbing material. By use of a liquid absorbing material, scattered liquid medicines, oral bacterial and other liquids are absorbed by the main sheet 10 and retained inside the main sheet 10. As a result, it is possible to prevent the patient's clothing from being soiled and various germs contained in liquids from contacting and entering the eyes and so on of the patient being treated. In this case, the side of the liquid absorbing material coming into contact with the patient being treated is preferably waterproofed and/or coated with a waterproof membrane. By this, it is possible to effectively absorb scattering liquid medicines and other liquids such as oral bacterial and to prevent permeating liquids from contacting the patient's clothing and body. Although paper and non-woven fabric are usable as the material of the main sheet 10, as are rayon and polyester-based fibers, the material is not limited to these.

The main sheet 10 is comprises the face covering portion 30. As shown in FIG. 1a, FIG. 1b and FIG. 2a, the face covering portion 30 is a member for covering the face of the patient being treated. The face covering portion 30 covers the face of the patient and, by protecting the face (especially the eyes) against matter scattered during treatment, plays a role in preventing bacterial contamination and infection.

In view of its purpose, the face covering portion 30 is preferably around a size that covers the whole face of the person being treated. In this embodiment, the lateral width of the face covering portion 30 is made about 350 to 400 mm and the vertical width about 250 to 300 mm, but this is not a limitation and the size can be chosen in line with the build of the patient. Further, although the shape of the face covering portion 30 is approximately rectangular in this embodiment, this is not a limitation, and it is possible to choose from various shapes, such as semicircular, elliptical and other shapes matched to the shape of the face, animal and popular character face shapes, and so on.

As seen in FIG. 1a, FIG. 1b and FIG. 2a, the opening 40 is a mouth-like opening cut in the middle of the face covering portion 30 for use during treatment, and is provided for performing oral cavity treatment of a patient wearing the surgical apron sheet according to this invention. The opening 40 is cut at a location corresponding to the mouth of the person being treated when the face covering portion 30 is placed on the face of the patient.

As shown in FIG. 1a and FIG. 1b, the opening 40 has a configuration provided with an engaging portion 42 by cutting its lower end in a substantially semielliptic shape so as to engage the mentum or lower jaw line of the person being treated. As shown in FIG. 2a, adoption of this configuration enables treatment by exposing only the patient's oral cavity through the opening 40 when the face covering portion 30 is placed on the patient during treatment, and forms a shape by which the engaging portion 42 of the lower end engages the submental region or lower jaw of the patient being treated and the mentum or jaw stays in the opening 40 even when the patient makes a big mouth movement. Owing to this configuration, the lower end of the opening engaging the underside of the mentum or the lower jaw line does not detach even when the mouth opens widely during treatment, so that there is no risk of the end of the drape/apron sheet entering the mouth or covering and impairing visibility of the oral cavity during treatment. Moreover, also in the case of the patient being treated opening and closing the mouth, the drape/apron sheet follows the movement of the mentum or lower jaw, so that a situation that makes interruption of treatment unavoidable owing to concealment of the patient's mouth by the face covering portion 30 can be reliably prevented.

As an arrangement is realized wherein the mentum or chin is fitted in the opening 40 thanks to the engaging portion 42, any gap between the face covering portion 30 and the face of the patient being treated is minimized, and expansion of the gap becomes almost nil even when the patient makes mouth movements during treatment. This makes it possible to reduce the risk of scattering liquid medicines and other liquids from infiltrating through the gap and further enables communication between the surgeon and the patient to proceed easily and safely even during treatment.

For immobilizing the face covering portion 30 of the main sheet 10, the opening 40 has a configuration provided at the upper end with a nose bridge engaging portion 44 for engaging along the nose bridge of the patient being treated. The nose bridge engaging portion 44 is a slit and/or cutout provided at the upper end of the opening 40 and configured to branch left-right from the peak of the nose contour. The nose bridge engaging portions 44 is cut upward from the upper end of the opening 40.

As shown in FIGS. 1a and 1b, it is possible as an embodiment to constitute the nose bridge engaging portion 44 as a slit-shaped incision provided in a vertical straight line in the upper end of the opening 40, and when this nose bridge engaging portion 44 constituted as a vertical linear slit abuts the nose of the patient being treated, the face covering portion 30 is, as shown in FIG. 2a, separated left-right along the ridge of the nose by the slit incision of the nose bridge engaging portion 44.

Further, as shown in FIG. 5, it is possible as another embodiment to constitute the nose bridge engaging portion 44 in a cutout shape provided in the upper end of the opening 40. Moreover, the nose bridge engaging portion 44 can be configured by in addition to providing the cutout also providing a slit constituted as a vertical linear incision into the upper end of the cutout. When this nose bridge engaging portion 44 constituted of the cutout, or the nose bridge engaging portion 44 constituted of the cutout and slit combined, comes into contact with the nose of the patient being treated, the face covering portion 30 separates left-right along the ridge of the nose.

Owing to the provision of the nose bridge engaging portion 44, the face covering portion 30 comes to engage the nose bridge in a state branching left-right along the ridge of the nose, and the face covering portion 30 can continue to maintain the proper fitted position even if pulled in the chin direction (downward), thus enabling prevention of fit slipping. Further, as this engagement enables the face covering portion 30 to approach near enough to both cheeks to make contact, the gap between the face covering portion 30 and the face of the patient being treated can be minimized and the risk of scattering liquid medicines and other liquids infiltrating through the gap can be reduced.

Avoidance of an unpleasant feeling owing to adherence of liquid medicine or other liquid on the nose can be anticipated especially with the configuration provided with the slit at the upper end of the opening 40. Further, a slip preventing effect can be expected with the configuration provided with the cutout owing to secure engagement with the nose. In addition, with the configuration that combines the cutout and slit, the covering portion 30 fits still more snugly on the face of the patient being treated, so that an effect of preventing uncomfortable constraint or infection owing to scatter of medicinal and other liquids can be expected.

Shape and length of the slit of the nose bridge engaging portion 44 are not limited to the vertical straight line, and can instead be a slanted line along the nose or a triangle. With such a structure, the opening 40 engages along the nose bridge of the patient being treated.

In the present embodiment, the opening 40 measures around 90 to 100 mm in vertical width, but is not limited to this and can be suitably selected in accordance with the size of the patient's face, mentum and chin. And while the lateral width of the opening 40 is around 65 to 70 mm in the present embodiment, it is not limited to this and can be suitably selected in accordance with the shape and size of the patient's mouth, mentum and chin. In addition, in the present embodiment, the upper end of the opening 40 makes an angle of 90 degrees to the slit of the nose bridge engaging portion 44 and the upper end is square, as shown in FIG. 1a and FIG. 1b, for example, but the shape is not limited to this and, aside from the vertical straight line, can be trimmed to a triangular or semicircular shape.

The main sheet 10 can be provided with the trunk covering portion 20. The trunk covering portion 20 is a member for covering the trunk of the patient being treated that is provided at the lower edge of the face covering portion, as shown in FIG. 1b and FIG. 2a. By covering the trunk of the patient, the trunk covering portion 20 helps to protect the patient's skin, clothing and so on from matter scattered during treatment so as to prevent contamination by various germs and the like.

In view of its purpose, the trunk covering portion 20 is preferably around a size that covers from the shoulders to the abdomen of the person being treated. In this embodiment, the lateral width of the trunk covering portion 20 is made about 450 to 500 mm and the vertical width about 330 to 400 mm, but this is not a limitation and the size can be freely chosen in line with the build and figure of the patient for use by, for example, an adult, child, male or female. Further, although the shape of the trunk covering portion 20 is approximately rectangular in this embodiment, this is not a limitation, and various shapes are possible, such as trapezoidal, human trunk shape, animal shape, or popular character shape.

Before the start of treatment, the face covering portion 30 of the surgical apron sheet according to the present invention is folded into a state overlying the trunk covering portion 20, as shown in FIG. 2b. This makes it easy for the patient being treated to communicate with a physician. During treatment, the face covering portion 30 is unfolded to cover the face of the patient being treated, as shown in FIG. 2a. As the oral cavity of the patient is exposed through the opening 40 at this time, treatment is facilitated, and the face, particularly the eyes, of the patient being treated is protected against contamination by matter scattered in the course of treatment.

By constituting the trunk covering portion 20 wider than the face covering portion 30, it is possible to adopt a configuration formed with left-right shoulder portions 22a and 22b at the junction with the face covering portion 30. Since the shoulder portions 22a and 22b are formed at the left-right upper ends of the trunk covering portion 20, as shown in FIG. 1b, these portions can be fastened to the patient's clothing or the like by clips or other fastening means. This can prevent the surgical apron sheet from slipping and falling off.

Moreover, fitting straps 24a and 24b for fastening the main sheet 10 to the patient being treated can be formed on the shoulder portions 22a and 22b. The fitting straps 24a and 24b are strip-like members joined to the left-right shoulder portions 22a and 22b. When the surgical apron sheet is used, fastening to the clothing or the like of the patient being treated can be achieved by tying the fitting straps 24a and 24b extending from the left and right shoulder portions 22a and 22b behind the patient's neck. By this, the surgical apron sheet can be kept from falling off during treatment.

A configuration is possible that provides the fitting straps 24a and 24b integrally with the trunk covering portion 20 to be partially detachable. For example, as shown in FIG. 3, left and right side edges of the face covering portion 30 can be detachably constituted and these portions serve as the fitting straps 24a and 24b. Further, when long fitting straps 24a and 24b are required, the fitting straps 24a and 24b can be formed as shown in FIG. 4 by detachably configuring an upper end portion of the face covering portion 30 in addition to the left and right sides. In addition, a shape can also be adopted that forms a loop after detachment and is donned from over the head without tying as strings. This enables the fitting straps 24a and 24b or a looped strap to be easily formed, and also as regards manufacture of the surgical apron sheet, efficient manufacture of a good product with absolutely no occurrence of cutout (trimming) waste owing to difference in breadth between the shoulder portions and the face portion.

1-4. (canceled)

5. A surgical apron sheet of apron shape, comprising:
at least one surface is waterproofed or coated with a waterproof membrane;
a face covering portion to cover a face of a patient during a treatment and provided with a mouth-like opening to perform an oral cavity treatment;
wherein the mouth-like opening is provided at a lower end of the surgical apron sheet with an engaging portion cut to substantially semi-elliptic shape to engage a submental region or lower jaw line of the patient being treated to immobilize the face covering portion.

6. The surgical apron sheet according to claim 5, further comprising a trunk covering portion provided at a lower end of the face covering portion to cover a trunk of the patient being treated.

7. The surgical apron sheet according to claim 5, further comprising a nose bridge engaging portion constituted in a shape formed by a slit or cutout incised upward from an upper end of the mouth-like opening to separate left-right from a peak of a nose contour at the upper end portion, whereby the mouth-like opening engages along a nose bridge of the patient.

8. The surgical apron sheet according claim 6, wherein the trunk covering portion is formed with shoulder portions of greater breadth than the face covering portion and fitting straps to fasten the surgical apron sheet to a body of the patient, the fitting straps are integrally attached, partially detachably, to the shoulder portions.
